(19) United States
(12) Patent Application Publication
(10) Pub. No.: US 2024/0138771 A1
(43) Pub. Date: May 2, 2024

(54) PULSE MANIFESTATION DETERMINING METHOD

(71) Applicant: AUO Corporation, Hsinchu (TW)

(72) Inventors: Chien Cheng Wang, Hsinchu (TW); Jung-Teng Pan, Hsinchu (TW); Chin-Tang Chuang, Hsinchu (TW)

(73) Assignee: AUO Corporation, Hsinchu (TW)

(21) Appl. No.: 18/361,938

(22) Filed: Jul. 31, 2023

(30) Foreign Application Priority Data
Nov. 1, 2022 (TW) 111141490

(51) Int. Cl. A61B 5/00; A61B 5/024

(52) U.S. Cl. CPC A61B 5/7221; A61B 5/02444; A61B 5/4854

(57) ABSTRACT

A pulse manifestation determining method is provided. The pulse manifestation method includes following steps. A pulse signal for determination is obtained. A valid range of the pulse signal for determination is determined through identifying a reverse pulse. A pulse manifestation is determined based on the valid range.

### CROSS-REFERENCE TO RELATED APPLICATION

This application claims the priority benefit of Taiwan application serial no. 111141490, filed on Nov. 1, 2022. The entirety of the above-mentioned patent application is hereby incorporated by reference herein and made a part of this specification.

### BACKGROUND

### Technical Field

The disclosure relates to a determining method; more particularly, the disclosure relates to a pulse manifestation determining method.

### Description of Related Art

According to the conventional Chinese medicine pulse diagnosis, “Cun,” “Guan,” and “Chi” positions (also known as the three sections) on a wrist are pressed to observe the pulse manifestation. Based on the pulse manifestation of the three sections, physiological or pathological conditions of different organs and meridians are determined. Additionally, a Chinese medicine pulse meter, in the form of a wristband, is used to measure the pulse by applying a pressure sensor, and the measurement may be manually controlled by a mechanical knob to exert a pressure or electronically controlled by an external pressure, such as an air bag, whose operation manner is similar to that of a sphygmomanometer. However, the existing diagnosis method based on the pulse manifestation relies on a standardized approach and fails to account for individual differences. As a result, the existing diagnosis method is unable to provide an accurate diagnosis based on the pulse manifestation determining result.

### SUMMARY

The disclosure provides a pulse manifestation determining method that may reduce the impact of individual differences to provide effective pulse manifestation diagnosis information.

According to an embodiment of the disclosure, a pulse manifestation determining method is provided and includes following steps. A pulse signal for determination is obtained. A valid range of the pulse signal for determination is determined through identifying a reverse pulse. A pulse manifestation is determined based on the valid range.

In view of the above, the pulse manifestation determining method provided in one or more embodiments of the disclosure relies on the valid range determined by the valid pulse of the pulse signal for determination, and the valid range serves as a basis to determine the pulse manifestation of the pulse signal for determination, rather than making determination based on numerical values including amplitude or pressure to be compared with a threshold for determination. Therefore, the pulse manifestation determining method provided in one or more embodiments of the disclosure may reduce the impact of individual differences and thus provide the effective pulse manifestation diagnosis information.

Several exemplary embodiments accompanied with figures are described in detail below to further describe the disclosure in details.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a flowchart of a pulse manifestation determining method according to an embodiment of the disclosure.

FIG. 2 is a flowchart of a pulse manifestation determining method according to another embodiment of the disclosure.

FIG. 3 is a schematic view illustrating a sensor disposed on a wrist according to an embodiment of the disclosure.

FIG. 4 is a schematic view illustrating waveforms of pulse signals for determining a pulse signal for determination according to an embodiment of the disclosure.

FIG. 5 is a schematic diagram view illustrating a waveform of a pulse signal for determination according to an embodiment of the disclosure.

FIG. 6 is a schematic view illustrating a waveform of a pulse signal for determination according to an embodiment of the disclosure, and a noise filtering operation is performed on the pulse signal for determination.

FIG. 7 is a schematic view illustrating a waveform of a pulse signal for determination according to an embodiment of the disclosure, and a noise filtering operation and an abnormal peak interval filtering operation are performed on the pulse signal for determination.

FIG. 8 is a schematic view illustrating a waveform of a pulse signal for determination according to an embodiment of the disclosure, and a noise filtering operation and an abnormal peak interval filtering operation are performed on the pulse signal for determination to filter a reverse pulse.

FIG. 9 is a schematic view illustrating a waveform for determining a normal pulse according to an embodiment of the disclosure.

FIG. 10 is a schematic view illustrating a waveform for determining a reverse pulse according to an embodiment of the disclosure.

FIG. 11 is a detailed flowchart illustrating the step S130 in FIG. 1 according to an embodiment of the disclosure.

FIG. 12 is a schematic view illustrating a waveform for determining a pulse manifestation based on a valid range according to an embodiment of the disclosure.

### DESCRIPTION OF THE EMBODIMENTS

Unless otherwise defined, all terminologies (including technical and scientific terminologies) used herein have the same meaning as commonly understood by people having ordinary skill in the art to which the disclosure belongs. It is understood that these terminologies, such as those defined in commonly used dictionaries, should be interpreted as having meanings consistent with the relevant art and the background or context of the disclosure, and should not be interpreted in an idealized or overly formal way, unless otherwise defined in the embodiments of the disclosure.

It should be understood that, although the terminologies “first,” “second,” “third,” and so forth may serve to describe various elements, components, regions, layers, and/or sections in this disclosure, these elements, components, regions, layers, and/or sections shall not be limited by these terminologies. These terminologies merely serve to distinguish one element, component, region, layer, and/or section from another element, component, region, layer, or section. Thus, a first “element,” “component,” “region,” “layer,” or “section” discussed below may be called as a second element, component, region, layer, or section without departing from the teachings herein.

The terminologies used herein are only for the purpose of describing particular embodiments and are not restrictive. As used herein, the singular forms “a,” “an,” and “the” are intended to include the plural forms including “at least one” or represent “and/or” unless the content clearly indicates otherwise. As used herein, the terminology “and/or” includes any and all combinations of one or more of the associated listed items. It should also be understood that when used in this disclosure, the terminologies “include” and/or “comprise” indicate the presence of the described features, regions, overall scenarios, steps, operations, elements, and/or components but do not exclude the presence or addition of one or more other features, regions, overall scenarios, steps, operations, elements, components, and/or combinations thereof.

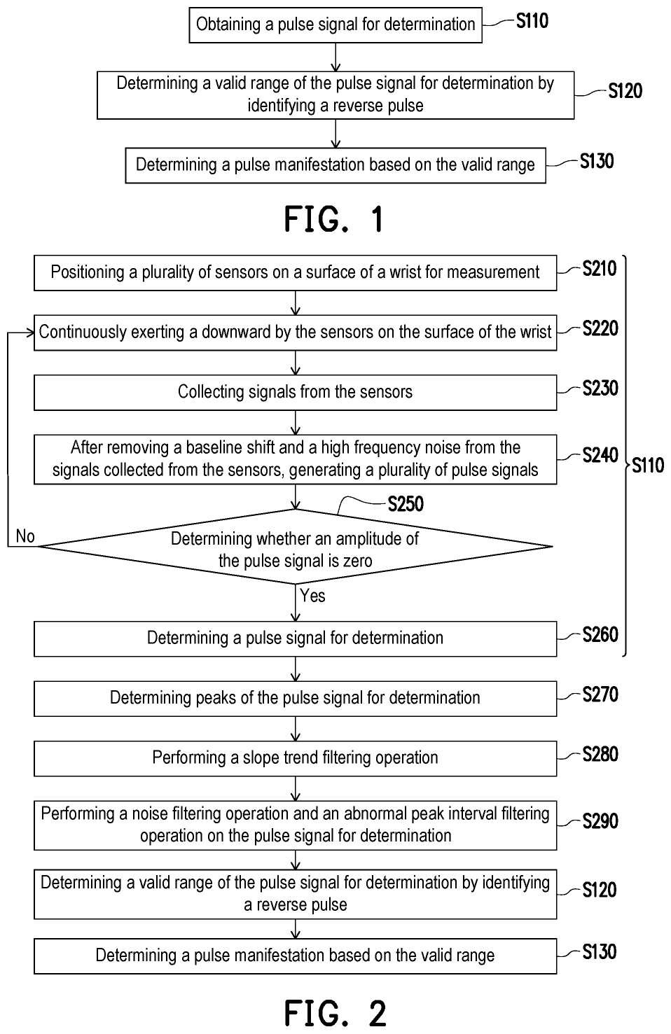

FIG. 1 is a flowchart of a pulse manifestation determining method according to an embodiment of the disclosure. With reference to FIG. 1, in this embodiment, a pulse manifestation determining method includes at least following steps. In step S110, a pulse signal for determination is obtained. In step S120, a valid range of the pulse signal for determination is determined by identifying a reverse pulse. In other words, the valid range of a valid pulse in the pulse signal for determination may be determined by filtering out the reverse pulse in the pulse signal for determination. In step S130, a pulse manifestation is determined based on the valid range. To be specific, since the pulse manifestations in traditional Chinese medicine are divided into a “floating pulse,” a “middle pulse,” and a “sunken pulse,” the valid range may be divided into three parts corresponding to the “floating pulse,” the “middle pulse,” and the “sunken pulse,” and the pulse manifestation presented by (or corresponding to) the pulse signal for determination may be determined according to pulse characteristics (such as the highest pulse peak) of the valid pulse in the filtered pulse signal for determination. Thereby, since the pulse manifestation is determined based on the valid range according to one or more embodiments of the disclosure, rather than making determination based on numerical values including amplitude or pressure to be compared with a threshold for determination, the impact of individual differences may be reduced, so as to provide effective pulse manifestation diagnosis information.

In an embodiment of the disclosure, some steps in the pulse manifestation determining method may be executed by a computer device, which may include a processor (e.g., a central processing unit (CPU), a graphics processing unit (GPU), an application processor (AP), a tensor processing Unit (TPU), or any other similar device) and a storage device storing commands (e.g., a random access memory (RAM), a read only memory (ROM), and so on). Moreover, the processor may execute the commands stored in the storage device to perform at least some steps in the pulse manifestation determining method.

FIG. 2 is a flowchart of a pulse manifestation determining method according to another embodiment of the disclosure. With reference to FIG. 1 and FIG. 2, in this embodiment, step S110 may be further divided into steps S210-S260, and steps S270-S290 are further added between steps S110 and S120.

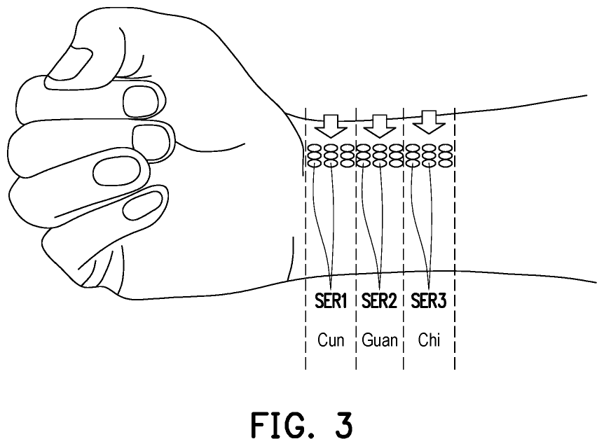

FIG. 3 is a schematic view illustrating a sensor disposed on a wrist according to an embodiment of the disclosure. With reference to FIG. 2 and FIG. 3, in step S210, a plurality of sensors SER1-SER3 are positioned on a surface of a wrist for measurement, where a set of sensors (such as the sensors SER1-SER3) are placed respectively at “Cun,” “Guan,” and “Chi” positions of the wrist to measure the pulse manifestations at these positions. Each set of sensors (such as the SER1-SER3) may be arranged in a 3×3 array, which should however not be construed as a limitation in the disclosure, and any number of array arrangements is applicable herein. After that, through various mechanisms or manual methods, the sensors (such as the sensors SER1-SER3) continuously exert a downward pressure on the surface of the wrist (step S220), and at the same time signals are collected from the sensors (such as the sensors SER1-SER3) (step S230). In this embodiment of the disclosure, a mechanical mechanism may be used to apply a pressure, thus allowing the sensors (such as the sensors SER1-SER3) to continuously exert a downward pressure at a constant speed.

After the signals are collected from the sensors (such as the sensors SER1-SER3), a baseline shift and a high frequency noise are removed from the signals collected from the sensors (such as the sensors SER1-SER3), and then a plurality of pulse signals for determining the pulse are generated (step S240). Next, it is determined whether an amplitude of the measured (or generated) pulse signals is zero (step S250). When the amplitude of the measured (or generated) pulse signals is not zero, i.e., the determination result is “no”, it indicates that the measurement process is not yet completed (nor ended), and the operation returns to step S220; when the amplitude of the measured (or generated) pulse signals is zero, it indicates that the measurement process has been completed (or ended); i.e., the determination result is “yes”, and then step S260 is executed. Namely, the sensors may continue to exert a pressure until the pulse amplitude cannot be measured.

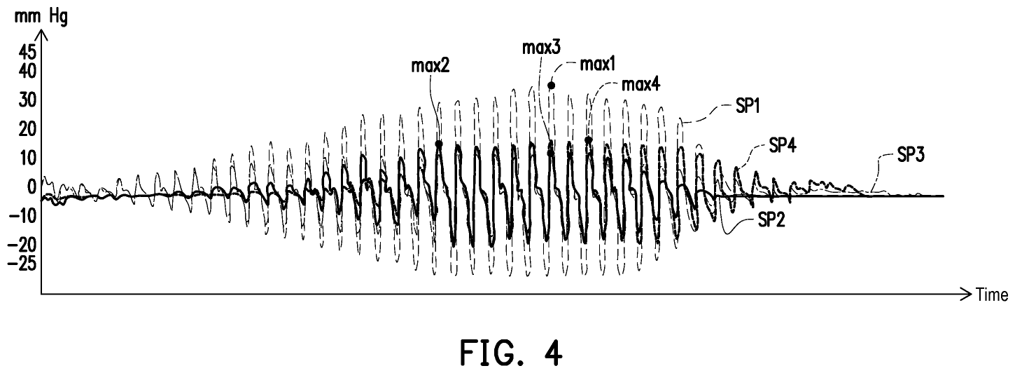

FIG. 4 is a schematic view illustrating waveforms of pulse signals for determining a pulse signal for determination according to an embodiment of the disclosure. With reference to FIG. 2 and FIG. 4, in step S260, the pulse signals are collected from the sensors (such as the sensors SER1-SER3) to determine a pulse signal for determination for determining the pulse manifestation. As shown in FIG. 4, waveform characteristics of exemplary pulse signals SP1-SP4 are determined (here, the highest peaks max1-max4 are taken for example), and based on the waveform characteristics of the pulse signals SP1-SP4, it is determined which one of the pulse signals SP1-SP4 is to be used as the pulse signal for determination (here, the pulse signal SP1 corresponding to the highest peak max1 with the highest value serves as the pulse signal for determination). Moreover, in this embodiment of the disclosure, a corresponding pulse signal for determination may be selected from the pulse signals corresponding to the “Cun” position, the “Guan” position, and the “Chi” position.

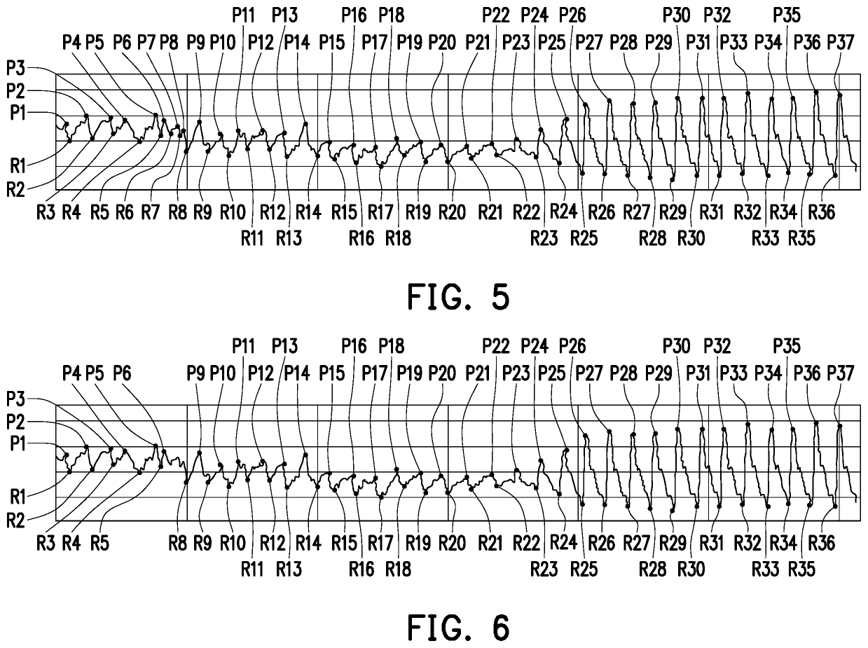

FIG. 5 is a schematic view illustrating a waveform of a pulse signal for determination according to an embodiment of the disclosure. With reference to FIG. 2 and FIG. 6, in step S270, peaks (such as P1-P37) and valleys (such as R1-R36) of the pulse signal for determination are detected. Then, as shown in FIG. 2, step S280 is executed to perform a slope trend filtering operation, so as to initially modify the waveform of the pulse signal for determination.

FIG. 6 is a schematic view illustrating a waveform of a pulse signal for determination according to an embodiment of the disclosure, and a noise filtering operation is performed on the pulse signal for determination. With reference to FIG. 2 and FIG. 6, in step S290, a noise filtering operation and an abnormal peak interval filtering operation are performed on the pulse signal for determination. Generally, pulses with overly small amplitudes may be considered as noise and directly filtered out; for instance, pulses with amplitudes less than 2 millimeters of mercury (mmHg) are considered as noise and directly filtered out, or pulses with amplitudes less than a certain proportion (e.g., 0.2) of the highest peak (such as the highest peak max1 shown in FIG. 4) are considered as noise and directly filtered out. As shown in FIG. 6, the peaks P7 and P8 and the valleys R6 and R7 are removed from the figure.

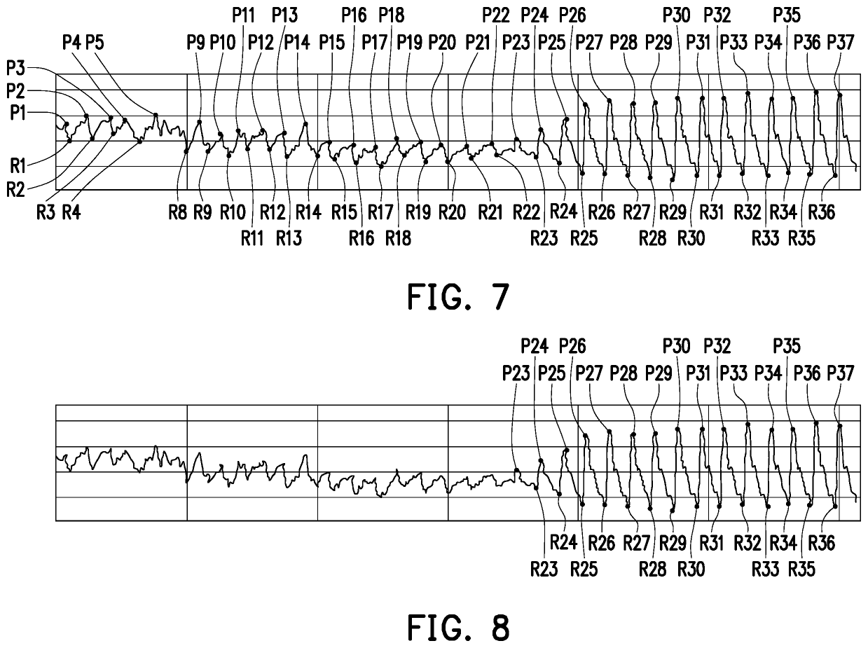

FIG. 7 is a schematic view illustrating a waveform of a pulse signal for determination according to an embodiment of the disclosure, and a noise filtering operation and an abnormal peak interval filtering operation are performed on the pulse signal for determination. With reference to FIG. 2 and FIG. 7, after the noise filtering operation is performed, intervals that are overly short may be considered as abnormal peak intervals, and the corresponding peaks and valleys are removed. As shown in FIG. 7, the peak P6 and the valley R5 are removed from the figure. The overly short intervals may be determined based on a pulse count (or a heartbeat count), e.g., half of the reciprocal of the pulse count, which may be determined based on the system design and should however not be construed as a limitation in the disclosure.

FIG. 8 is a schematic view illustrating a waveform of a pulse signal for determination according to an embodiment of the disclosure, and a noise filtering operation and an abnormal peak interval filtering operation are performed on the pulse signal for determination to filter a reverse pulse. With reference to FIG. 2 and FIG. 8, in step S290, after the noise filtering operation and the abnormal peak interval filtering operation are performed on the pulse signal for determination, a valid range of the pulse signal for determination is further determined by identifying a reverse pulse. Specifically, the reverse pulse in the pulse signal for determination is firstly identified (such as the peaks P1-P5 and P9-21 and the valleys R1-R4 and R9-R22), and the reverse pulse in the pulse signal for determination is removed (such as the peaks P1-P5 and P9-21 and the valleys R1-R4 and R9-R22), and the remaining pulses in the pulse signal for determination are considered as valid pulses (such as the peaks P23-37 and the valleys R23-R36). Finally, the valid range of the pulse signal for determination may be determined based on the valid pulses in the pulse signal for determination (such as the peaks P23-37 and the valleys R23-R36). For instance, the first valid pulse peak (such as the peak P23) and the last valid pulse peak (such as the peak P37) in the pulse signal for determination may serve to define the valid range of the pulse signal for determination.

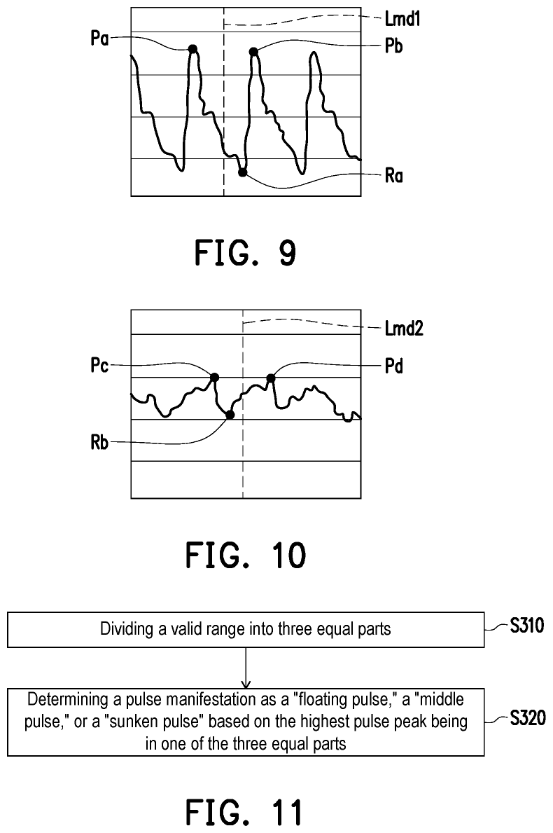

FIG. 9 is a schematic view illustrating a waveform for determining a normal pulse according to an embodiment of the disclosure. With reference to FIG. 2, FIG. 8, and FIG. 9, in a normal pulse, a valley Ra is closer to the next peak Pb than a previous peak Pa; that is, an x-axis position (i.e., a time point) of the valley Ra is greater than (or later than) an x-axis position of a midline Lmd1 between the two peaks Pa and Pb. In other words, in the pulse signal for determination, when a current valley (for instance, the valley Ra) located between the previous peak (for instance, the peak Pa) and the current peak (for instance, the peak Pb) is closer to the previous peak (for instance, the peak Pa), the current valley (for instance, the valley Ra) and the current peak (for instance, the peak Pb) constitute a reverse pulse.

FIG. 10 is a schematic view illustrating a waveform for determining a reverse pulse according to an embodiment of the disclosure. With reference to FIG. 2, FIG. 8, and FIG. 10, in a reverse pulse, a valley Rb is closer to the previous peak Pc than the next peak Pd; that is, an x-axis position (i.e., a time point) of the valley Rb is less than (or earlier than) an x-axis position of the midline Lmd2 between the two peaks Pc and Pd. In other words, in the pulse signal for determination, when the current valley (for instance, the valley Rb) located between the previous peak (for instance, the peak Pc) and the current peak (for instance, the peak Pd) is closer to the current peak (for instance, the peak Pd), the current valley (for instance, the valley Rb) and the current peak (for instance, the peak Pd) constitute a normal pulse.

FIG. 11 is a detailed flowchart illustrating the step S130 in FIG. 1 according to an embodiment of the disclosure. With reference to FIG. 2 and FIG. 11, in this embodiment, step S130 may include steps S310 and S320. In step S310, the valid range is divided into three equal parts, and in step S320, the pulse manifestation is determined as a “floating pulse,” a “middle pulse,” or a “sunken pulse” based on the highest pulse peak being in one of the three equal parts.

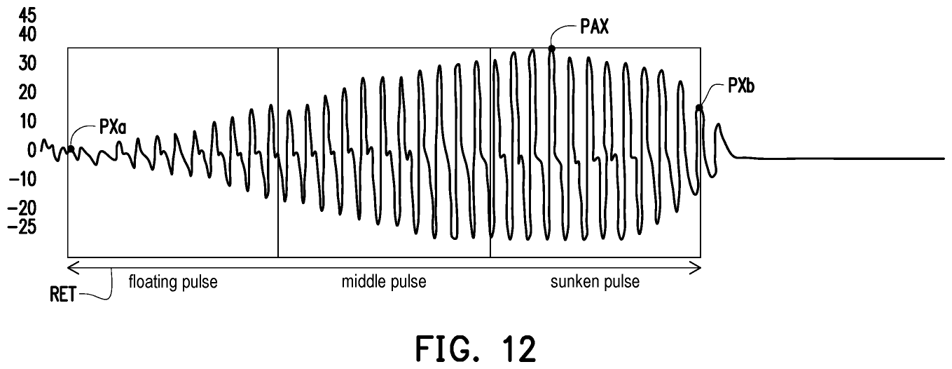

FIG. 12 is a schematic view illustrating a waveform for determining a pulse manifestation based on a valid range according to an embodiment of the disclosure. With reference to FIG. 11 and FIG. 12, after determining a valid range RET based on a first valid pulse peak PXa and a last valid pulse peak PXb, the valid range RET is divided into three equal parts corresponding to the “floating pulse,” the “middle pulse,” and the “sunken pulse”, respectively. The pulse manifestation of the pulse signal for determination is then determined as the “floating pulse,” the “middle pulse,” or the “sunken pulse” based on which of the three equal parts a highest pulse peak PMX of the pulse signal for determination is located. In this embodiment, the highest pulse peak PMX is located in the range corresponding to the “sunken pulse,” so that the pulse manifestation of the pulse signal for determination is determined as the “sunken pulse. By the same token, when the highest pulse peak PMX is located in the range corresponding to the “middle pulse,” the pulse manifestation of the pulse signal for determination may be determined as the “middle pulse”; when the highest pulse peak PMX is located in the range corresponding to the “floating pulse,” the pulse manifestation of the pulse signal for determination may be determined as the “floating pulse”.

As described in the previous embodiments, the pulses may be recorded in real time during a pressing process of the sensors at a constant speed according to one or more embodiments of the disclosure, and whether these pulses are valid is determined, so as to clearly define start and end points of the valid pulses. After that, based on the start point and the end point of the valid pulses, the valid range for determining the pulse manifestation is defined, and the valid range is automatically segmented corresponding to the “floating pulse,” the “middle pulse,” and the “sunken pulse,” so as to effectively determine the pulse manifestation of the pulse signal for determination.

To sum up, the pulse manifestation determining method provided in one or more embodiments of the disclosure is applied to determine the valid range based on the valid pulses of the pulse signal for determination, and the valid range is applied to determine the pulse manifestation of the pulse signal for determination, rather than making determination based on numerical values including amplitude or pressure to be compared with a threshold for determination. Therefore, the pulse manifestation determining method provided in one or more embodiments of the disclosure may reduce the impact of individual differences, so as to provide effective pulse manifestation diagnosis information.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A pulse manifestation determining method, comprising:
obtaining a pulse signal for determination;
determining a valid range of the pulse signal for determination through identifying a reverse pulse; and
determining a pulse manifestation based on the valid range.

2. The pulse manifestation determining method according to claim 1, further comprising:
receiving a plurality of pulse signals from a plurality of sensors to determine the pulse signal for determination.

3. The pulse manifestation determining method according to claim 2, further comprising:
selecting a corresponding pulse signal for determination from the pulse signals corresponding to “Cun,” “Guan,” and “Chi” positions.

4. The pulse manifestation determining method according to claim 2, wherein these sensors are arranged in an array.

5. The pulse manifestation determining method according to claim 2, wherein during a measurement process, the sensors continuously exert a pressure until a pulse amplitude is unable to be measured.

6. The pulse manifestation determining method according to claim 2, further comprising:
generating the pulse signals after removing a baseline shift and a high-frequency noise from the signals received by the sensors.

7. The pulse manifestation determining method according to claim 1, wherein the step of determining the pulse manifestation based on the valid range comprises:
dividing the valid range into three equal parts; and
determining the pulse manifestation as a “floating pulse,” a “middle pulse,” or a “sunken pulse” based on a highest pulse peak located in one of the three equal parts.

8. The pulse manifestation determining method according to claim 1, further comprising:
before determining the valid range of the pulse signal for determination through identifying the reverse pulse, performing a noise filtering operation and an abnormal peak interval filtering operation on the pulse signal for determination.

9. The pulse manifestation determining method according to claim 8, further comprising:
defining the valid range of the pulse signal for determination based on a first valid pulse peak and a last valid pulse peak of the pulse signal for determination.

10. The pulse manifestation determining method according to claim 1, further comprising:
when a current valley located between a previous peak and a current peak in the pulse signal for determination is closer to the previous peak, the current valley and the current peak form a reverse pulse, and
when the current valley is closer to the current peak, the current valley and the current peak form a normal pulse.
